(19) United States
(12) Patent Application Publication
(10) Pub. No.: US 2005/0267070 A1
(43) Pub. Date: Dec. 1, 2005

(54) Process for the preparation of esters of heparin

(75) Inventors: Lorenzo De Ferra, Rome (IT); Mauro Montorsi, Modena (IT)

Correspondence Address: DARBY & DARBY P.C., P. O. BOX 5257, NEW YORK, NY, 10150-5257, US

(73) Assignee: CHEMI SPA, Milano (IT)

(21) Appl. No.: 11/195,392

(22) Filed: Aug. 1, 2005

Related U.S. Application Data
(63) Continuation of application No. 10/402,330, filed on Mar. 28, 2003, now abandoned.
(60) Provisional application No. 60/437,310, filed on Dec. 31, 2002.

(30) Foreign Application Priority Data
Jun. 20, 2002 (EP) 02077534.2

(51) Int. Cl. A61K 31/727; C08B 37/10

(52) U.S. Cl. 514/56; 536/21

(57) ABSTRACT

The description relates to a process for the production of esters of heparin, wherein from 0.1 to 2 g of a halogenated reagent having the formula R—CH₂—X, where R is a phenyl group which is non-substituted or substituted by a halogen atom or by a nitro group, and X is a halogen atom, preferably chlorine, are reacted with from 2 to 20 g of one of the quaternary ammonium salts of heparin in from 30 to 250 ml of N,N-dimethylformamide and/or N,N-dimethylacetamide. The process in question allows esters of heparin to be obtained at lower cost and within shorter times than the methods known in the art, minimising among other things the use of lachrymatory reagents, such as, for example, benzyl chloride.

The present invention relates to a process for the preparation of esters of heparin, wherein carboxylic groups are esterified partially or totally with a R—CH₂-radical, where R is preferably a non-substituted, or substituted phenyl group, by reaction with a halogenated reagent having the formula R—CH₂—X in N,N-dimethylformamide at a temperature of between 35° and 90° C. for a reaction time of between 1 and 20 hours.

### PRIOR ART

The present invention relates to a process for the preparation of esters of heparin which are useful as intermediates in the synthesis of heparin having a low molecular weight, in particular of Enoxaparin (Common International Denomination—CID), a principal active ingredient with anti-coagulant and anti-thrombotic action, whose preparation is described in European patent application EP-40144 and in American U.S. Pat. No. 5,389,618, both incorporated herein by reference.

The therapeutic use of Enoxaparin is, in particular, described in detail in various scientific publications, such as, for example, Frydman et al., J. Clin. Pharmacol., 1988; 28:609-618; Planes et al., Haemostasis, 16:152-158, 1986; Dahan et al., 16:152-158, 1986; Huet et al., 16:152-158, 1986; Aiach et al., Thrombosis Res., 31:611-621, 1983; Neville et al., Journal of Pharmaceutical Sciences, Vol. 78, no. 2, February 1989; Neville et al., Journal of Pharmaceutical Sciences, Vol. 79, no. 4, April 1990.

It is known from the prior art, in particular from European patent application EP-40144 and from American U.S. Pat. No. 4,440,926, to prepare esters of heparin which are derived by the partial or total esterification of the carboxylic groups which are present in the structure of heparin; the esters in question are intermediates useful in the preparation, via alkaline hydrolysis with consequent de-polymerisation, of heparins having low molecular weight, such as, for example, Enoxaparin, which have pharmacological advantages over the basic heparin, in particular as regards a substantial reduction in the anti-coagulant activity, and therefore in the haemorrhagic risk, which is greatly feared above all in surgery.

In greater detail, EP-40144 and U.S. Pat. No. 4,440,926 describe the partial or complete esterification of the carboxylic groups which are present in the polymer chain of the heparin, by introducing the group R—CH₂by treatment of a quaternary ammonium salt of heparin with a large stoichiometric excess, of from 10 to 30 times the amount necessary to esterify the carboxylic groups present sent in the polymer chain of the heparin, of a halogenated reagent having the formula R—CH₂—X, where R can have a number of meanings, R preferably being a phenyl group, non-substituted or substituted by a halogen atom or by a nitro group, and X is a halogen atom, preferably a chlorine atom; the halogenated reagent is preferably benzyl chloride.

The esterification reaction described in the prior art cited is carried out with the large excess of halogenated reagent for a very long period of time, from 24 to 72 hours, usually at ambient temperature. Large quantities of solvent, normally in the order of 20 ml of solvent per gramme of quaternary ammonium salt of heparin, are further used in EP-40144 and U.S. Pat. No. 4,440,926.

This process for esterifying heparin which is carried out according to the teaching of patents EP-40144 and U.S. Pat. No. 4,440,926 leads to substantial disadvantages from the economic, industrial and environmental points of view in that both the substantial excess of the solvent and that of the halogenated agent increase the production costs considerably, make the separation of the excess of halogenated reagent and the purification of the ester of heparin difficult and costly, and make the disposal of the reaction refluents costly and environmentally harmful. Furthermore, the long reaction times, of from 24 to 72 hours, constitute a very negative factor of the use of the installations from the economic/industrial point of view.

A further significant problem of the processes described in EP-40144 and U.S. Pat. No. 4,440,926 is linked to the fact that the halogenated reagents having the formula R—CH₂—X, such as, for example, benzyl chloride, are strong lachrymatory agents; their use in large quantities in processes on an industrial scale therefore poses significant problems from the point of view of safety at work.

### DESCRIPTION OF THE INVENTION

Therefore, the object of the present invention is to provide a process for the production of esters of heparin, which can be used for the subsequent production of Enoxaparin, according to the teaching of European patent application EP 40144 and American U.S. Pat. No. 5,389,618, which is free from the above-mentioned disadvantages.

In particular, the subject-matter of the present invention allows all of the serious negative aspects which are present in the above-described prior art to be overcome by providing an industrially economic and environmentally sound process for the preparation of esters of heparin which are useful as intermediates in the synthesis of heparin having low molecular weight, which is universally known as Enoxaparin.

The subject-matter of the present invention consists in a process for the preparation of esters of heparin, wherein carboxylic groups are esterified partially or completely by means of the partial or complete substitution of the hydrogen atoms or of the cations which salify the carboxylic groups by the radical R—CH₂—, where R represents a hydrogen atom or an aliphatic or aromatic group which is substituted or non-substituted, preferably by a halogen atom or by a nitro group.

This preparation is effected by reacting from 0.1 to 2 parts by weight of a halogenated reagent having the formula R—CH₂—X, where R is a phenyl group which is non-substituted or substituted by a halogen atom or by a nitro group, and X is a halogen atom, preferably chlorine, with from 2 to 20 parts by weight of one of the quaternary ammonium salts of heparin in from 30 to 250 parts by volume of an inert organic solvent selected from N,N-dimethylformamide, N,N-dimethylacetamide and methylene chloride.

For the purposes of the present invention, the above-described parts by weight are to be understood to be in grammes and the parts by volume in millilitres. Therefore, according to a preferred feature of the invention, from 0.5 to 1.5 g of halogenated reagent are reacted with from 8 to 16 g of quaternary ammonium salt in from 60 to 150 ml of solvent; even more preferably, from 0.8 to 1.2 g of halogenated reagent are reacted with from 10 to 14 g of quaternary ammonium salt in from 80 to 120 ml of solvent.

According to a feature of the invention, a quantity of a quaternary ammonium salt of heparin such as to contain a stoichiometric equivalent of carboxylic groups is reacted with from 0.6 to 1.5 stoichiometric equivalents of a halogenated reagent having the formula R—CH₂—X, where R has the above-mentioned meaning and X represents a halogen atom, in an inert solvent at a temperature of between 35° C. and 90° C. for a reaction time of between 1 and 8 hours and the ester so obtained is precipitated from the reaction medium, cooled to ambient temperature by the addition of a solution of sodium acetate in methyl alcohol.

The above-mentioned quaternary ammonium salt of heparin can be produced according to one of the processes which are already known in the art, such as precisely those described in European patent application EP-40144 and in American U.S. Pat. No. 5,389,618; in turn, the heparin used for the production of the quaternary ammonium salt can be a common commercially available heparin which meets the requirements of the Official Pharmacopoeia. In a preferred feature of the invention, the esterification reaction is carried out by reacting from 0.8 to 1.1 stoichiometric equivalents of halogenated reagent R—CH₂—X, where R is a phenyl group which is non-substituted or substituted by a chlorine atom or a nitro group in the para position, and X is a chlorine atom, with a quantity of benzethonium salt of heparin such as to contain a stoichiometric equivalent of carboxylic groups in from 5 to 15 volumes, in respect of the weight of the heparin salt, in inert solvent selected lected from N,N-dimethylformamide, N,N-dimethylacetamide and/or methylene chloride.

In a more preferred feature of the invention, R represents non-substituted phenyl, the reaction solvent is N,N-dimethylformamide, the temperature is between 65° C. and 75° C. and the reaction time is between 1 and 3 hours.

Upon completion of the reaction, the mixture is cooled to ambient temperature and the ester of heparin is precipitated by the addition of a 10%-solution of sodium acetate in methyl alcohol.

The esters so obtained have chemical/physical features which are similar to those of esters prepared according to the cited prior art and a heparin having low molecular weight and the features corresponding to those of Enoxaparin (CID) which are listed in the European Official Pharmacopoeia is obtained therefrom by following the process of hydrolysis/de-polymerisation described in the examples of European patent application EP-40144 and American U.S. Pat. No. 5,389,618.

The examples referred to below are a further non-limiting illustration of the invention.

### EXAMPLE 1

15 grammes of benzethonium salt of heparin were dissolved in 150 ml of N,N-dimethylformamide and there were added to the solution 0.79 ml of benzyl chloride (d=1.1 purity 99%) and the reaction mixture was heated to 70° C. for two hours. The reaction mixture was then cooled to ambient temperature and the ester was precipitated by the addition of 300 ml of a 10%-solution of sodium acetate in methyl alcohol.

After filtration, the solid was washed with methyl alcohol and dried under vacuum; 5.01 g of benzyl ester of heparin were obtained.

### EXAMPLE 2

The esterification reaction was carried out according to the same method described in example 1, the sole change being the use of 0.83 ml of benzyl chloride. 5.04 g of benzyl ester of heparin were obtained.

### EXAMPLE 3

The esterification reaction was carried out according to the same method described in example 1, the sole change being the use of 0.96 ml of benzyl chloride. 5.03 g of benzyl ester of heparin were obtained.

### EXAMPLE 4

A 21-flask was filled under nitrogen with 60 g of neutral heparin benzethonium salt which has been prepared beforehand and 480 ml of dimethylformamide; the mixture was stirred at ambient temperature until dissolution was complete. 4.63 g of benzyl chloride were added; the mixture was heated with stirring to approximately 50° C. The temperature and stirring were maintained for approximately 12 hours until a check by sampling during the reaction established that the reaction was complete. The mixture was cooled to ambient temperature. A solution prepared beforehand of 96 g of anhydrous sodium acetate in 960 ml of methanol was then added slowly; the temperature and stirring were maintained for 30 minutes. The wet product was filtered and put back into the flask, and 340 ml of methanol were added. The mixture was stirred for 1 hour at ambient temperature ture and was then filtered and washed with 80 ml of methanol. The wet product was then dried at reduced pressure and approximately 25 g of heparin benzyl ester sodium salt were obtained.

The ester so obtained has then been subjected to a depolymerisation treatment as described in the examples appended to European patent application EP-40144, obtaining from 5 to 7 g of Enoxaparin conforming to European Pharmacopoeia.

1. A process for the production of esters of heparin, said process comprising the step of reacting
(a) from 0.1 to 2 parts by weight of a halogenated reagent having the formula R—CH₂—X, where R is a phenyl group which is non-substituted or substituted by a halogen atom or by a nitro group, and X is a halogen atom, and
(b) from 8 to 20 parts by weight of a quaternary ammonium salt of heparin, said reaction occurring in from 30 to 250 parts by volume of an inert organic solvent selected from the group consisting of N,N-dimethylformamide, N,N-dimethylacetamide and methylene chloride.

2. The process of claim 1, wherein from 0.5 to 1.5 parts by weight of the halogenated reagent are reacted with from 8 to 16 parts by weight of the quaternary ammonium salt in from 60 to 150 parts by volume of the inert organic solvent.

3. The process of claim 2, wherein from 0.8 to 1.2 parts by weight of the halogenated reagent is reacted with from 10 to 14 parts by weight of the quaternary ammonium salt, in from 80 to 120 parts by volume of the inert organic solvent.

4. The process of claim 1, wherein X is chlorine.

5. The process of claim 3, wherein 1 part by weight of the halogenated reagent is used.

6. The process of claim 3, wherein 12 parts by weight of quaternary ammonium salt is used.

7. The process of claim 3, wherein 100 parts by volume of the inert organic solvent is used.

8. The process of claim 3, wherein a quantity of quaternary ammonium salt of heparin containing a stoichiometric equivalent of carboxylic groups is reacted with between 0.6 and 1.5 stoichiometric equivalents, of the halogenated reagent.

9. The process of claim 8, wherein between 0.6 and 1.5 stoichiometric equivalents of halogenated reagent is used.

10. The process of claim 3, wherein the quaternary ammonium salt of heparin is the benzethonium salt of heparin.

11. The process of claim 3, wherein the halogenated reagent is selected from the group consisting of benzyl chloride, 4-chlorobenzyl chloride and 4-nitobenzyl chloride.

12. The process of claim 3, which is carried out in N,N-dimethyformamide.

13. The process of claim 3, which is carried out at a temperature of between 35° C. and 90° C.

14. The process of claim 13, which is carried out at a temperature of between 40° C. and 75° C.

15. The process of claim 14, which is carried out at a temperature of between 45° C. and 60° C.

16. The process of claim 14, wherein the reaction is carried out for a period of time of between 1 hour and 20 hours.

17. The process of claim 14, wherein the reaction is carried out for a period of time of between 1 hour and 16 hours.

18. The process of claim 17, wherein the reactions is carried out for a period of time between 1 and 3 hours.

19. The process of claim 3, wherein the reaction product is precipitated from the reaction medium by the addition of a solution of sodium acetate in methyl alcohol.

20. A process for a production of enoxaparin, said process for the production of esters of heparin according to claim 3.
